(19) United States
(12) Patent Application Publication
(10) Pub. No.: US 2012/0226018 A1
(43) Pub. Date: Sep. 6, 2012

(54) Process for the Synthesis of Ac-Arg-Cyclo(Cys-D-Ala-His-D-Phe-Arg-Trp-Cys)-NH2

(75) Inventor: Zheng Xin Dong, Holliston, MA (US)

(73) Assignee: IPSEN PHARMA, S.A.S., Boulogne-Billancourt (FR)

(21) Appl. No.: 13/508,786

(22) PCT Filed: Nov. 15, 2010
(86) PCT No.: PCT/US10/56696

§ 371 Date: May 9, 2012

Related U.S. Application Data
(60) Provisional application No. 61/281,359, filed on Nov. 16, 2009.

(51) Int. Cl. C07K 7/64

(52) U.S. Cl. 530/317

(57) ABSTRACT

The present invention relates to a novel process for the synthesis of the melanocortin analog, Ac-Arg-cyclo(Cys-D-Ala-His-D-Phe-Arg-Trp-Cys)-NH₂, using solution-phase peptide chemistry.

### BACKGROUND OF THE INVENTION

The present invention relates to a novel process for the synthesis of the melanocortin analog, Ac-Arg-cyclo(Cys-D-Ala-His-D-Phe-Arg-Trp-Cys)-NH₂, using solution-phase peptide chemistry. Ac-Arg-cyclo(Cys-D-Ala-His-D-Phe-Arg-Trp-Cys)-NH₂ is a highly potent and pharmacologically selective analog of melanocortin. Melanocortins are a family of regulatory peptides which are formed by post-translational processing of pro-hormone pro-opiomelanocortin (POMC; 131 amino acids in length). POMC is processed into three classes of hormones; the melanocortins, adrenocorticotropin hormone, and various endorphins (e.g. lipotropin) (Cone, et al., Recent Prog. Horm. Res., 51:287-317, (1996); Cone et al., Ann. N.Y. Acad. Sci., 31:342-363, (1993)).

Melanocortins have been found in a wide variety of normal human tissues including the brain, adrenal, skin, testis, spleen, kidney, ovary, lung, thyroid, liver, colon, small intestine and pancreas (Tatro, J. B. et al., Endocrinol. 121:1900-1907 (1987); Mountjoy, K. G. et al., Science 257:1248-1251 (1992); Chhajlani, V. et al., FEBS Lett. 309:417-420 (1992); Gantz, I. et al. J. Biol. Chem. 268:8246-8250 (1993) and Gantz, I. et al., J. Biol. Chem. 268:15174-15179 (1993)).

Melanocortin peptides have been shown to exhibit a wide variety of physiological activities including the control of behavior and memory, affecting neurotrophic and antipyretic properties, as well as affecting the modulation of the immune system. Aside from their well known effects on adrenal cortical functions (adrenocorticotropic hormone or “ACTH”) and on melanocytes (melanocyte stimulating hormone or “MSH”), melanocortins have also been shown to control the cardiovascular system, analgesia, thermoregulation and the release of other neurohumoral agents including prolactin, luteinizing hormone and biogenic amines (De Wied, D. et al., Methods Achiev. Exp. Pathol. 15:167-199 (1991); De Wied, D. et al., Physiol. Rev. 62:977-1059 (1982); Guber, K. A. et al., Am. J. Physiol. 257:R681-R694 (1989); Walker J. M. et al., Science 210:1247-1249 (1980); Murphy, M. T. et al., Science 221:192-193 (1983); Ellerkmann, E. et al., Endocrinol. 130:133-138 (1992) and Versteeg, D. H. G. et al., Life Sci. 38:835-840 (1986)).

It has also been shown that binding sites for melanocortins are distributed in many different tissue types including lachrymal and submandibular glands, pancreas, adipose, bladder, duodenum, spleen, brain and gonadal tissues as well as malignant melanoma tumors. Five melanocortin receptors have been characterized to date. These include melanocyte-specific receptor (MC1-R), corticoadrenal-specific ACTH receptor (MC2-R), melacortin-3 (MC3-R), melanocortin-4 (MC4-R) and melanocortin-5 receptor (MC5-R). All of the melanocortin receptors respond to the peptide hormone class of melanocyte stimulating hormones (MSH) (Cone, R. D. et al., Ann. N.Y. Acad. Sci., 680:342-363 (1993); Cone, R. D. et al., Recent Prog. Horm. Res., 51:287-318 (1996)).

MC1-R, known in the art as Melanocyte Stimulating Hormone Receptor (MSH-R), Melanotropin Receptor or Melanocortin-1 Receptor, is a 315 amino acid transmembrane protein belonging to the family of G-Protein coupled receptors. MC1-R is a receptor for both MSH and ACTH. The activity of MC1-R is mediated by G-proteins which activate adenylate cyclase. MC1-R receptors are found in melanocytes and corticoadrenal tissue as well as various other tissues such as adrenal gland, leukocytes, lung, lymph node, ovary, testis, pituitary, placenta, spleen and uterus. MC2-R, also called Adrenocorticotropic Hormone Receptor (ACTH-R), is a 297 amino acid transmembrane protein found in melanocytes and the corticoadrenal tissue. MC2-R mediates the corticotrophic effect of ACTH. In humans, MC3-R is a 360 AA protein found in brain tissue; in mice and rats MC3-R is a 323 AA protein. MC4-R is a 332 amino acid transmembrane protein which is also expressed in brain as well as placental and gut tissues. MC5-R is a 325 amino acid transmembrane protein expressed in the adrenals, stomach, lung and spleen and very low levels in the brain. MC5-R is also expressed in the three layers of adrenal cortex, predominantly in the aldosterone-producing zona glomerulosa cells.

The five known melanocortin receptors differ, however, in their functions. For example,

MC1-R is a G-protein coupled receptor that regulates pigmentation in response to α-MSH, a potent agonist of MC1-R. Agonism of the MC1-R receptor results in stimulation of the melanocytes which causes eumelanin and increases the risk for cancer of the skin. Agonism of MC1-R can also have neurological effects. Stimulation of MC2-R activity can result in carcinoma of adrenal tissue. Recent pharmacological confirmation has established that central MC4-R receptors are the prime mediators of the anorexic and orexigenic effects reported for melanocortin agonists and antagonists, respectively. The effects of agonism of the MC3-R and MC5-R are not yet known.

There has been great interest in melanocortin receptors as targets for the design of novel therapeutics to treat disorders of body weight such as obesity and cachexia. Both genetic and pharmacological evidence points toward central MC4-R receptors as the principal target (Giraudo, S. Q. et al., Brain Res., 809:302-306 (1998); Farooqi, I. S. et al., NE J Med., 348:1085-1095 (2003); MacNeil, D. J. et al., Eu. J. Pharm., 44:141-157 (2002); MacNeil, D. J. et al., Eu. J. Pharm., 450:93-109 (2002); Kask, A. et al., NeuroReport, 10:707-711 (1999)). The current progress with receptor-selective agonists and antagonists evidences the therapeutic potential of melanocortin receptor activation, particularly MC4-R.

The solution-phase synthesis described in U.S. Pat. No. 4,395,403 uses BTFA/TFA to remove the methoxybenzyl group protecting the thiol group of cysteine followed by cyclization. Decomposition of tryptophan residue, however, is known to frequently occur during such harsh acid treatment for removal of protecting groups. As such, there is a need for developing an efficient method for producing Ac-Arg-cyclo(Cys-D-Ala-His-D-Phe-Arg-Trp-Cys)-NH₂.

### SUMMARY OF THE INVENTION

In a first aspect of the present invention, there is provided a novel process for the synthesis of Ac-Arg-cyclo(Cys-D-Ala-His-D-Phe-Arg-Trp-Cys)-NH₂, which comprises a fragment condensation procedure, wherein protected amino acids, such as Boc protected amino acids, benzoyloxycarbonyl protected amino acids, Fmoc protected amino acids, and protected amino acid fluorides, such as Fmoc amino acid fluoride or Bsmoc amino acid fluoride, are used, wherein a mixed anhydride coupling method is employed, and wherein a protected peptide fragment Trp-Cys or Arg-Trp, such as Boc-Trp-Cys(Acm)-OMe or Boc-Trp(For)-Cys(Acm)-OMe, is provided.

In a preferred embodiment of the first aspect of the present invention, a peptide-hydrazide coupling method is employed, wherein ammonia is used to convert an ester functional group to an amide functional group.

The first aspect of the present invention may comprise the steps of:

(a) synthesizing a fragment benzoyloxycarbonyl-D-Ala-His-OH from benzoyloxycarbonyl-D-Ala-OH and H-His-OH in the presence of a coupling reagent;

or alternatively, synthesizing a fragment benzoyloxycarbonyl-D-Ala-His(Trt)-OH from benzoyloxycarbonyl-D-Ala-OH and H-His(Trt)-OH in the presence of a coupling reagent;

(b-1) synthesizing a fragment benzoyloxycarbonyl-D-Phe-Arg(Pbf)-OMe from benzoyloxycarbonyl-D-Phe-OH and H-Arg(Pbf)-OMe in the presence of a coupling reagent;

(b-2) synthesizing a fragment H-D-Phe-Arg(Pbf)-OMe by hydrogenating the fragment benzoyloxycarbonyl-D-Phe-Arg(Pbf)-OMe obtained in the step (b-1);

(c-1) synthesizing a fragment benzoyloxycarbonyl-D-Ala-His-D-Phe-Arg(Pbf)-OMe from benzoyloxycarbonyl-D-Ala-His-OH and the fragment H-D-Phe-Arg(Pbf)-OMe obtained in the step (b-2) in the presence of a coupling reagent;

or alternatively, synthesizing a fragment benzoyloxycarbonyl-D-Ala-His(Trt)-D-Phe-Arg(Pbf)-OMe from benzoyloxycarbonyl-D-Ala-His(Trt)-OH and the fragment H-D-Phe-Arg(Pbf)-OMe obtained in the step (b-2) in the presence of a coupling reagent;

(c-2) synthesizing a fragment H-D-Ala-His-D-Phe-Arg(Pbf)-OMe by hydrogenating the fragment benzoyloxycarbonyl-D-Ala-His-D-Phe-Arg(Pbf)-OMe obtained in the step (c-1);

or alternatively, synthesizing a fragment H-D-Ala-His(Trt)-D-Phe-Arg(Pbf)-OMe by hydrogenating the fragment benzoyloxycarbonyl-D-Ala-His(Trt)-D-Phe-Arg(Pbf)-OMe obtained in the step (c-1);

(d-1) synthesizing a fragment Fmoc-Arg(Pbf)-Cys(Acm)-OH from Fmoc-Arg(Pbf)-OH and H-Cys(Acm)-OBzl in the presence of a coupling reagent, followed by hydrogenation;

(d-2) synthesizing a fragment H-Arg(Pbf)-Cys(Acm)-OH from the fragment Fmoc-Arg(Pbf)-Cys(Acm)-OH obtained in the step (d-1) in the presence of a base;

(d-3) synthesizing a fragment Ac-Arg(Pbf)-Cys(Acm)-OH from the fragment H-Arg(Pbf)-Cys(Acm)-OH obtained in the step (d-2);

or alternatively, synthesizing a fragment Ac-Arg(Pbf)-Cys(Acm)-OH from Ac-Arg(Pbf)-OH and H-Cys(Acm)-OMe in the presence of a coupling reagent, followed by hydrolysis by using a base;

(e-1) synthesizing a fragment Ac-Arg(Pbf)-Cys(Acm)-D-Ala-His-D-Phe-Arg(Pbf)-OMe from Ac-Arg(Pbf)-Cys(Acm)-OH and H-D-Ala-His-D-Phe-Arg(Pbf)-OMe in the presence of a coupling reagent;

or alternatively, synthesizing a fragment Ac-Arg(Pbf)-Cys(Acm)-D-Ala-His(Trt)-D-Phe-Arg(Pbf)-OMe from Ac-Arg(Pbf)-Cys(Acm)-OH and H-D-Ala-His(Trt)-D-Phe-Arg(Pbf)-OMe in the presence of a coupling reagent;

(e-2) synthesizing a fragment Ac-Arg(Pbf)-Cys(Acm)-D-Ala-His-D-Phe-Arg(Pbf)-OH by hydrolyzing the fragment Ac-Arg(Pbf)-Cys(Acm)-D-Ala-His-D-Phe-Arg(Pbf)-OMe obtained in the step (e-1) in the presence of a base;

or alternatively, synthesizing a fragment Ac-Arg(Pbf)-Cys(Acm)-D-Ala-His(Trt)-D-Phe-Arg(Pbf)-OH by hydrolyzing the fragment Ac-Arg(Pbf)-Cys(Acm)-D-Ala-His(Trt)-D-Phe-Arg(Pbf)-OMe obtained in the step (e-1) in the presence of a base;

(f) synthesizing a fragment Ac-Arg(Pbf)-Cys(Acm)-D-Ala-His-D-Phe-Arg(Pbf)-NHNH₂ from the fragment Ac-Arg(Pbf)-Cys(Acm)-D-Ala-His-D-Phe-Arg(Pbf)-OMe obtained in the step (e-1) in the presence of hydrazine;

or alternatively, synthesizing a fragment Ac-Arg(Pbf)-Cys(Acm)-D-Ala-His(Trt)-D-Phe-Arg(Pbf)-NHNH₂ from the fragment Ac-Arg(Pbf)-Cys(Acm)-D-Ala-His(Trt)-D-Phe-Arg(Pbf)-OMe obtained in the step (e-1) in the presence of hydrazine;

(g-1) synthesizing a fragment Boc-Trp-Cys(Acm)-OMe from Boc-Trp-OH and H-Cys(Acm)-OMe in the presence of a coupling reagent;

or alternatively, synthesizing a fragment Boc-Trp(For)-Cys(Acm)-OMe from Boc-Trp(For)-OH and H-Cys(Acm)-OMe in the presence of a coupling reagent;

(g-2) synthesizing H-Trp-Cys(Acm)-OMe from the fragment Boc-Trp-Cys(Acm)-OMe obtained in the step (g-1) in the presence of TFA;

or alternatively, synthesizing H-Trp(For)-Cys(Acm)-OMe from the fragment Boc-Trp(For)-Cys(Acm)-OMe obtained in the step (g-1) in the presence of TFA;

(h) synthesizing Ac-Arg(Pbf)-Cys(Acm)-D-Ala-His-D-Phe-Arg(Pbf)-Trp(For)-Cys(Acm)-OMe from the fragment Ac-Arg(Pbf)-Cys(Acm)-D-Ala-His-D-Phe-Arg(Pbf)-NHNH₂ obtained in the step (f) and H-Trp(For)-Cys(Acm)-OMe by using an acid and tert-butylnitrite;

or alternatively, synthesizing Ac-Arg(Pbf)-Cys(Acm)-D-Ala-His(Trt)-D-Phe-Arg(Pbf)-Trp(For)-Cys(Acm)-OMe from the fragment Ac-Arg(Pbf)-Cys(Acm)-D-Ala-His(Trt)-D-Phe-Arg(Pbf)-NHNH₂ obtained in the step (f) and H-Trp(For)-Cys(Acm)-OMe by using an acid and tert-butylnitrite;

or alternatively, synthesizing Ac-Arg(Pbf)-Cys(Acm)-D-Ala-His-D-Phe-Arg(Pbf)-Trp-Cys(Acm)-OMe from the fragment Ac-Arg(Pbf)-Cys(Acm)-D-Ala-His-D-Phe-Arg(Pbf)-NHNH₂ obtained in the step (f) and H-Trp-Cys(Acm)-OMe by using an acid and tert-butylnitrite;

or alternatively, synthesizing Ac-Arg(Pbf)-Cys(Acm)-D-Ala-His(Trt)-D-Phe-Arg(Pbf)-Trp-Cys(Acm)-OMe from the fragment Ac-Arg(Pbf)-Cys(Acm)-D-Ala-His(Trt)-D-Phe-Arg(Pbf)-NHNH₂ obtained in the step (f) and H-Trp-Cys(Acm)-OMe by using an acid and tert-butylnitrite;

or alternatively, synthesizing Ac-Arg(Pbf)-Cys(Acm)-D-Ala-His-D-Phe-Arg(Pbf)-Trp-Cys(Acm)-OMe from Ac-Arg(Pbf)-Cys(Acm)-D-Ala-His-D-Phe-Arg(Pbf)-OH and H-Trp-Cys(Acm)-OMe in the presence of a coupling reagent;

or alternatively, synthesizing Ac-Arg(Pbf)-Cys(Acm)-D-Ala-His(Trt)-D-Phe-Arg(Pbf)-Trp(For)-Cys(Acm)-OMe from Ac-Arg(Pbf)-Cys(Acm)-D-Ala-His(Trt)-D-Phe-Arg(Pbf)-OH and H-Trp(For)-Cys(Acm)-OMe in the presence of a coupling reagent;

or alternatively, synthesizing Ac-Arg(Pbf)-Cys(Acm)-D-Ala-His-D-Phe-Arg(Pbf)-Trp(For)-Cys(Acm)-OMe from Ac-Arg(Pbf)-Cys(Acm)-D-Ala-His-D-Phe-Arg(Pbf)-OH and H-Trp(For)-Cys(Acm)-OMe in the presence of a coupling reagent;

or alternatively, synthesizing Ac-Arg(Pbf)-Cys(Acm)-D-Ala-His(Trt)-D-Phe-Arg(Pbf)-Trp-Cys(Acm)-OMe from Ac-Arg(Pbf)-Cys(Acm)-D-Ala-His(Trt)-D-Phe-Arg(Pbf)-OH and H-Trp-Cys(Acm)-OMe in the presence of a coupling reagent;

or alternatively, synthesizing Ac-Arg-Cys(Acm)-D-Ala-His-D-Phe-Arg-Trp-Cys(Acm)-OMe from Ac-Arg(Pbf)-Cys(Acm)-D-Ala-His-D-Phe-Arg(Pbf)-Trp-Cys(Acm)-OMe in the presence of TFA;

or alternatively, synthesizing Ac-Arg-Cys(Acm)-D-Ala-His-D-Phe-Arg-Trp-Cys(Acm)-OMe from Ac-Arg(Pbf)-Cys(Acm)-D-Ala-His(Trt)-D-Phe-Arg(Pbf)-Trp-Cys(Acm)-OMe in the presence of TFA;

or alternatively, synthesizing Ac-Arg-Cys(Acm)-D-Ala-His-D-Phe-Arg-Trp(For)-Cys(Acm)-OMe from Ac-Arg(Pbf)-Cys(Acm)-D-Ala-His-D-Phe-Arg(Pbf)-Trp(For)-Cys(Acm)-OMe in the presence of TFA;

or alternatively, synthesizing Ac-Arg-Cys(Acm)-D-Ala-His-D-Phe-Arg-Trp(For)-Cys(Acm)-OMe from Ac-Arg(Pbf)-Cys(Acm)-D-Ala-His(Trt)-D-Phe-Arg(Pbf)-Trp(For)-Cys(Acm)-OMe in the presence of TFA;

(i) synthesizing Ac-Arg(Pbf)-Cys(Acm)-D-Ala-His-D-Phe-Arg(Pbf)-Trp-Cys(Acm)-NH₂ from Ac-Arg(Pbf)-Cys(Acm)-D-Ala-His-D-Phe-Arg(Pbf)-Trp-Cys(Acm)-OMe obtained in the step (h) in the presence of ammonia;

or alternatively, synthesizing Ac-Arg-Cys(Acm)-D-Ala-His-D-Phe-Arg-Trp-Cys(Acm)-NH₂ from Ac-Arg-Cys(Acm)-D-Ala-His-D-Phe-Arg-Trp-Cys(Acm)-OMe obtained in the step (h) in the presence of ammonia;

or alternatively, synthesizing Ac-Arg-Cys(Acm)-D-Ala-His-D-Phe-Arg-Trp-Cys(Acm)-NH₂ from Ac-Arg-Cys(Acm)-D-Ala-His-D-Phe-Arg-Trp(For)-Cys(Acm)-OMe obtained in the step (h) in the presence of ammonia;

or alternatively, synthesizing Ac-Arg(Pbf)-Cys(Acm)-D-Ala-His(Trt)-D-Phe-Arg(Pbf)-Trp-Cys(Acm)-NH₂ from Ac-Arg(Pbf)-Cys(Acm)-D-Ala-His(Trt)-D-Phe-Arg(Pbf)-Trp-Cys(Acm)-OMe obtained in the step (h) in the presence of ammonia;

or alternatively, synthesizing Ac-Arg-Cys(Acm)-D-Ala-His-D-Phe-Arg-Trp-Cys(Acm)-NH₂ from Ac-Arg(Pbf)-Cys(Acm)-D-Ala-His-D-Phe-Arg(Pbf)-Trp-Cys(Acm)-NH₂ in the presence of TFA;

or alternatively, synthesizing Ac-Arg-Cys(Acm)-D-Ala-His-D-Phe-Arg-Trp-Cys(Acm)-NH₂ from Ac-Arg(Pbf)-Cys(Acm)-D-Ala-His(Trt)-D-Phe-Arg(Pbf)-Trp-Cys(Acm)-NH₂ in the presence of TFA; and

(j) synthesizing Ac-Arg-cyclo(Cys-D-Ala-His-D-Phe-Arg-Trp-Cys)-NH₂ from Ac-Arg-Cys(Acm)-D-Ala-His-D-Phe-Arg-Trp-Cys(Acm)-NH₂ by using an oxidizing agent.

In the above-described aspect of the present invention, said oxidizing agent preferably is iodine, said coupling agent preferably is DCC, HBTU, HATU, DIC, EDC, or chloroformic acid isobutyl ester, and said base preferably is Et₂NH, TAEA, piperazine, sodium hydroxide, or potassium hydroxide.

The synthetic sequences summarized above are schematically diagrammed in FIG. 1A.

FIG. 1B shows another schematic diagram of the synthetic sequences summarized above.

In a second aspect of the present invention, there is provided a novel process for the synthesis of Ac-Arg-cyclo(Cys-D-Ala-His-D-Phe-Arg-Trp-Cys)-NH₂ which is similar to the first aspect described above, except a protected peptide fragment Cys-D-Ala or Arg-Cys is provided.

In a preferred embodiment of the second aspect of the present invention, said protected peptide fragment Cys-D-Ala is Boc-Cys(Acm)-D-Ala-OH.

The second aspect of the present invention may comprise the steps of:

(a-1) synthesizing a fragment Boc-Cys(Acm)-D-Ala-OH from Boc-Cys(Acm)-OH and H-D-Ala-OH in the presence of a coupling reagent;

(a-2) synthesizing a fragment H-Cys(Acm)-D-Ala-OH from the fragment Boc-Cys(Acm)-D-Ala-OH obtained in the step (a-1) in the presence of TFA;

(b) synthesizing a fragment Ac-Arg(Pbf)-Cys(Acm)-D-Ala-OH from Ac-Arg(Pbf)-OH and the fragment H-Cys(Acm)-D-Ala-OH obtained in the step (a-2) in the presence of a coupling reagent;

(c-1) synthesizing a fragment Boc-His-D-Phe-OMe from Boc-His-OH and H-D-Phe-OMe in the presence of a coupling reagent;

(c-2) synthesizing a fragment H-His-D-Phe-OMe from the fragment Boc-His-D-Phe-OMe obtained in the step (c-1) in the presence of TFA;

(d-1) synthesizing a fragment Boc-Trp-Cys(Acm)-OMe from Boc-Trp-OH and H-Cys(Acm)-OMe in the presence of a coupling reagent;

or alternatively, synthesizing a fragment Boc-Trp(For)-Cys(Acm)-OMe from Boc-Trp(For)-OH and H-Cys(Acm)-OMe in the presence of a coupling reagent;

(d-2) synthesizing H-Trp-Cys(Acm)-OMe from the fragment Boc-Trp-Cys(Acm)-OMe obtained in the step (d-1) in the presence of TFA;

or alternatively, synthesizing H-Trp(For)-Cys(Acm)-OMe from the fragment Boc-Trp(For)-Cys(Acm)-OMe obtained in the step (d-1) in the presence of TFA;

(e-1) synthesizing a fragment benzoyloxycarbonyl-Arg(Pbf)-Trp-Cys(Acm)-OMe from benzoyloxycarbonyl-Arg(Pbf)-OH and the fragment H-Trp-Cys(Acm)-OMe obtained in the step (d-2) in the presence of a coupling reagent;

or alternatively, synthesizing a fragment benzoyloxycarbonyl-Arg(Pbf)-Trp(For)-Cys(Acm)-OMe from benzoyloxycarbonyl-Arg(Pbf)-OH and the fragment H-Trp(For)-Cys(Acm)-OMe obtained in the step (d-2) in the presence of a coupling reagent;

(e-2) synthesizing a fragment H-Arg(Pbf)-Trp-Cys(Acm)-OMe by hydrogenating the fragment benzoyloxycarbonyl-Arg(Pbf)-Trp-Cys(Acm)-OMe obtained in the step (e-1);

or alternatively, synthesizing a fragment H-Arg(Pbf)-Trp(For)-Cys(Acm)-OMe by hydrogenating the fragment benzoyloxycarbonyl-Arg(Pbf)-Trp(For)-Cys(Acm)-OMe obtained in the step (e-1);

(f-1) synthesizing a fragment Ac-Arg(Pbf)-Cys(Acm)-D-Ala-His-D-Phe-OMe from the fragment Ac-Arg(Pbf)-Cys(Acm)-D-Ala-OH obtained in the step (b) and the fragment H-His-D-Phe-OMe obtained in the step (c-2) in the presence of a coupling reagent;

(f-2) synthesizing a fragment Ac-Arg(Pbf)-Cys(Acm)-D-Ala-His-D-Phe-NHNH₂ from the fragment Ac-Arg(Pbf)-Cys(Acm)-D-Ala-His-D-Phe-OMe obtained in the step (f-1) in the presence of hydrazine;

or alternatively, synthesizing a fragment Ac-Arg(Pbf)-Cys(Acm)-D-Ala-His-D-Phe-OH by hydrolizing the fragment Ac-Arg(Pbf)-Cys(Acm)-D-Ala-His-D-Phe-OMe obtained in the step (f-1) in the presence of a base;

(g) synthesizing Ac-Arg(Pbf)-Cys(Acm)-D-Ala-His-D-Phe-Arg(Pbf)-Trp-Cys(Acm)-OMe from the fragment Ac-Arg(Pbf)-Cys(Acm)-D-Ala-His-D-Phe-NHNH₂ obtained in the step (f-2) and the fragment H-Arg(Pbf)-Trp-Cys(Acm)-OMe obtained in the step (e-2) using an acid and tert-butylnitrite;

or alternatively, synthesizing Ac-Arg(Pbf)-Cys(Acm)-D-Ala-His-D-Phe-Arg(Pbf)-Trp(For)-Cys(Acm)-OMe from the fragment Ac-Arg(Pbf)-Cys(Acm)-D-Ala-His-D-Phe-NHNH₂ obtained in the step (f-2) and the fragment H-Arg(Pbf)-Trp(For)-Cys(Acm)-OMe obtained in the step (e-2) using an acid and tert-butylnitrite;

or alternatively, synthesizing Ac-Arg(Pbf)-Cys(Acm)-D-Ala-His-D-Phe-Arg(Pbf)-Trp-Cys(Acm)-OMe from the fragment Ac-Arg(Pbf)-Cys(Acm)-D-Ala-His-D-Phe-OH obtained in the step (f-2) and the fragment H-Arg(Pbf)-Trp-Cys(Acm)-OMe obtained in the step (e-2) in the presence of a coupling agent;

or alternatively, synthesizing Ac-Arg(Pbf)-Cys(Acm)-D-Ala-His-D-Phe-Arg(Pbf)-Trp(For)-Cys(Acm)-OMe from the fragment Ac-Arg(Pbf)-Cys(Acm)-D-Ala-His-D-Phe-OH obtained in the step (f-2) and the fragment H-Arg(Pbf)-Trp(For)-Cys(Acm)-OMe obtained in the step (e-2) in the presence of a coupling agent;

(h) synthesizing Ac-Arg(Pbf)-Cys(Acm)-D-Ala-His-D-Phe-Arg(Pbf)-Trp-Cys(Acm)-NH₂ from Ac-Arg(Pbf)-Cys(Acm)-D-Ala-His-D-Phe-Arg(Pbf)-Trp-Cys(Acm)-OMe obtained in the step (g) in the presence of ammonia;

or alternatively, synthesizing Ac-Arg(Pbf)-Cys(Acm)-D-Ala-His-D-Phe-Arg(Pbf)-Trp(For)-Cys(Acm)-NH₂ from Ac-Arg(Pbf)-Cys(Acm)-D-Ala-His-D-Phe-Arg(Pbf)-Trp(For)-Cys(Acm)-)OMe obtained in the step (g) in the presence of ammonia;

or alternatively, synthesizing Ac-Arg(Pbf)-Cys(Acm)-D-Ala-His-D-Phe-Arg(Pbf)-Trp-Cys(Acm)-NH₂ from Ac-Arg(Pbf)-Cys(Acm)-D-Ala-His-D-Phe-Arg(Pbf)-Trp-Cys(Acm)-OMe obtained in the step (g) in the presence of ammonia;

or alternatively, synthesizing Ac-Arg(Pbf)-Cys(Acm)-D-Ala-His-D-Phe-Arg(Pbf)-Trp(For)-Cys(Acm)-NH₂ from Ac-Arg(Pbf)-Cys(Acm)-D-Ala-His-D-Phe-Arg(Pbf)-Trp(For)-Cys(Acm)-OMe obtained in the step (g) in the presence of ammonia;

(i) synthesizing Ac-Arg-Cys(Acm)-D-Ala-His-D-Phe-Arg-Trp-Cys(Acm)-NH₂ from Ac-Arg(Pbf)-Cys(Acm)-D-Ala-His-D-Phe-Arg(Pbf)-Trp-Cys(Acm)-NH₂ obtained in the step (h) in the presence of TFA; and

(j) synthesizing Ac-Arg-cyclo(Cys-D-Ala-His-D-Phe-Arg-Trp-Cys)-NH₂ from Ac-Arg-Cys(Acm)-D-Ala-His-D-Phe-Arg-Trp-Cys(Acm)-NH₂ obtained in the step (i) by using an oxidizing agent.

FIG. 2 is a schematic diagram of the synthetic sequences summarized immediately above.

FIGS. 3-11 are schematic diagrams of various synthetic sequences employing fragment condensation steps and different peptide fragments which all result in Ac-Arg-cyclo(Cys-D-Ala-His-D-Phe-Arg-Trp-Cys)-NH₂.

In a third aspect of the present invention, there is provided a novel process for the synthesis of Ac-Arg-cyclo(Cys-D-Ala-His-D-Phe-Arg-Trp-Cys)-NH₂ which comprises a linear stepwise synthetic procedure.

In a preferred embodiment of the third aspect of the present invention, protected amino acids, such as Boc protected amino acids, Fmoc protected amino acids, and protected amino acid fluorides, such as Fmoc amino acid fluoride or Bsmoc amino acid fluoride, are used, dimethylcyclopropylmethyl amine is used at the C-terminus of a protected peptide chain, and Fmoc-Cys(Trt)-NH-CMe₂CP is used.

The second aspect of the present invention may comprise the steps of:

(a) synthesizing H-Cys(Trt)-NH-CMe₂CP from Fmoc-Cys(Trt)-NH-CMe₂CP in the presence of a base;

(b) synthesizing Fmoc-Trp(Boc)-Cys(Trt)-NH-CMe₂CP from Fmoc-Trp(Boc)-OH and H-Cys(Trt)-NH-CMe₂CP obtained in the step (a) in the presence of a coupling reagent;

(c) synthesizing H-Trp(Boc)-Cys(Trt)-NH-CMe₂CP from Fmoc-Trp(Boc)-Cys(Trt)-NH-CMe₂CP obtained in the step (b) in the presence of a base;

(d) synthesizing Fmoc-Arg(Pbf)-Trp(Boc)-Cys(Trt)-NH-CMe₂CP from Fmoc-Arg(Pbf)-OH and H-Trp(Boc)-Cys(Trt)-NH-CMe₂CP obtained in the step (c) in the presence of a coupling reagent;

(e) synthesizing H-Arg(Pbf)-Trp(Boc)-Cys(Trt)-NH-CMe₂CP from Fmoc-Arg(Pbf)-Trp(Boc)-Cys(Trt)-NH-CMe₂CP obtained in the step (d) in the presence of a base;

(f) synthesizing Fmoc-D-Phe-Arg(Pbf)-Trp(Boc)-Cys(Trt)-NH-CMe₂CP from Fmoc-D-Phe-OH and H-Arg(Pbf)-Trp(Boc)-Cys(Trt)-NH-CMe₂CP obtained in the step (e) in the presence of a coupling reagent;

(g) synthesizing H-D-Phe-Arg(Pbf)-Trp(Boc)-Cys(Trt)-NH-CMe₂CP from Fmoc-D-Phe-Arg(Pbf)-Trp(Boc)-Cys(Trt)-NH-CMe₂CP obtained in the step (f) in the presence of a base;

(h) synthesizing Fmoc-His(Trt)-D-Phe-Arg(Pbf)-Trp(Boc)-Cys(Trt)-NH-CMe₂CP from Fmoc-His(Trt)-OH and H-D-Phe-Arg(Pbf)-Trp(Boc)-Cys(Trt)-NH-CMe₂CP obtained in the step (g) in the presence of a coupling reagent;

(i) synthesizing H-His(Trt)-D-Phe-Arg(Pbf)-Trp(Boc)-Cys(Trt)-NH-CMe₂CP from Fmoc-His(Trt)-D-Phe-Arg(Pbf)-Trp(Boc)-Cys(Trt)-NH-CMe₂CP obtained in the step (h) in the presence of a base;

(j) synthesizing Fmoc-D-Ala-His(Trt)-D-Phe-Arg(Pbf)-Trp(Boc)-Cys(Trt)-NH-CMe₂CP from Fmoc-D-Ala-OH and H-His(Trt)-D-Phe-Arg(Pbf)-Trp(Boc)-Cys(Trt)-NH-CMe₂CP obtained in the step (i) in the presence of a coupling reagent;

(k) synthesizing H-D-Ala-His(Trt)-D-Phe-Arg(Pbf)-Trp(Boc)-Cys(Trt)-NH-CMe₂CP from Fmoc-D-Ala-His(Trt)-D-Phe-Arg(Pbf)-Trp(Boc)-Cys(Trt)-NH-CMe₂CP obtained in the step (j) in the presence of a base;

(l) synthesizing Fmoc-Cys(Trt)-D-Ala-His(Trt)-D-Phe-Arg(Pbf)-Trp(Boc)-Cys(Trt)-NH-CMe₂CP from Fmoc-Cys(Trt)-OH and H-D-Ala-His(Trt)-D-Phe-Arg(Pbf)-Trp(Boc)-Cys(Trt)-NH-CMe₂CP obtained in the step (k) in the presence of a coupling reagent;

(m) synthesizing H-Cys(Trt)-D-Ala-His(Trt)-D-Phe-Arg(Pbf)-Trp(Boc)-Cys(Trt)-NH-CMe₂CP from Fmoc-Cys(Trt)-D-Ala-His(Trt)-D-Phe-Arg(Pbf)-Trp(Boc)-Cys(Trt)-NH-CMe₂CP obtained in the step (1) in the presence of a base;

(n) synthesizing Fmoc-Arg(Pbf)-Cys(Trt)-D-Ala-His(Trt)-D-Phe-Arg(Pbf)-Trp(Boc)-Cys(Trt)-NH-CMe₂CP from Fmoc-Arg(Pbf)-OH and H-Cys(Trt)-D-Ala-His(Trt)-D-Phe-Arg(Pbf)-Trp(Boc)-Cys(Trt)-NH-CMe₂CP obtained in the step (m) in the presence of a coupling reagent;

(o) synthesizing H-Arg(Pbf)-Cys(Trt)-D-Ala-His(Trt)-D-Phe-Arg(Pbf)-Trp(Boc)-Cys(Trt)-NH-CMe₂CP from Fmoc-Arg(Pbf)-Cys(Trt)-D-Ala-His(Trt)-D-Phe-Arg(Pbf)-Trp(Boc)-Cys(Trt)-NH-CMe₂CP obtained in the step (n) in the presence of a base;

(p) synthesizing Ac-Arg(Pbf)-Cys(Trt)-D-Ala-His(Trt)-D-Phe-Arg(Pbf)-Trp(Boc)-Cys(Trt)-NH-CMe₂CP from H-Arg(Pbf)-Cys(Trt)-D-Ala-His(Trt)-D-Phe-Arg(Pbf)-Trp(Boc)-Cys(Trt)-NH-CMe₂CP obtained in the step (o) and Ac₂O, AcCl or AcBr;

or alternatively, synthesizing Ac-Arg(Pbf)-Cys(Trt)-D-Ala-His(Trt)-D-Phe-Arg(Pbf)-Trp(Boc)-Cys(Trt)-NH-CMe₂CP from Ac-Arg(Pbf)-OH and H-Cys(Trt)-D-Ala-His(Trt)-D-Phe-Arg(Pbf)-Trp(Boc)-Cys(Trt)-NH-CMe₂CP obtained in the step (m) in the presence of a coupling reagent;

(q) synthesizing Ac-Arg-Cys-D-Ala-His-D-Phe-Arg-Trp-Cys-NH₂ from Ac-Arg(Pbf)-Cys(Trt)-D-Ala-His(Trt)-D-Phe-Arg(Pbf)-Trp(Boc)-Cys(Trt)-NH-CMe₂CP obtained in the step (p) in the presence of TFA; and

(r) synthesizing Ac-Arg-cyclo(Cys-D-Ala-His-D-Phe-Arg-Trp-Cys)-NH₂ from Ac-Arg-Cys-D-Ala-His-D-Phe-Arg-Trp-Cys-NH₂ obtained in the step (q) in the presence of an oxidizing agent.

In the third aspect of the present invention, said oxidizing agent preferably is iodine, oxygen, air, or DMSO; said coupling agent preferably is DCC, HBTU, HATU, DIC, EDC, or chloroformic acid isobutyl ester, and said base preferably is Et₂NH, TAEA, or piperazine.

### BRIEF DESCRIPTION OF THE DRAWINGS

FIG. 1A is a schematic diagram of the first aspect of the present invention as summarized above.

FIG. 1B shows another schematic diagram of the first aspect of the present invention as summarized above.

FIG. 2 is a schematic diagram of the second aspect of the present invention as summarized above.

FIGS. 3-11 are schematic diagrams of various synthetic sequences employing fragment condensation steps and different peptide fragments, which all result in Ac-Arg-cyclo(Cys-D-Ala-His-D-Phe-Arg-Trp-Cys)-NH₂, and which are within the scope of the present invention as claimed herein.

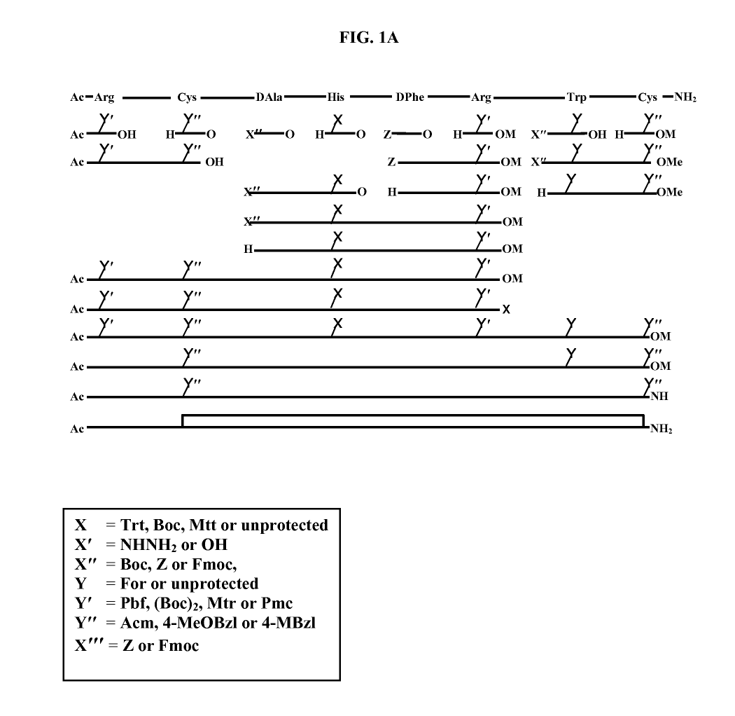

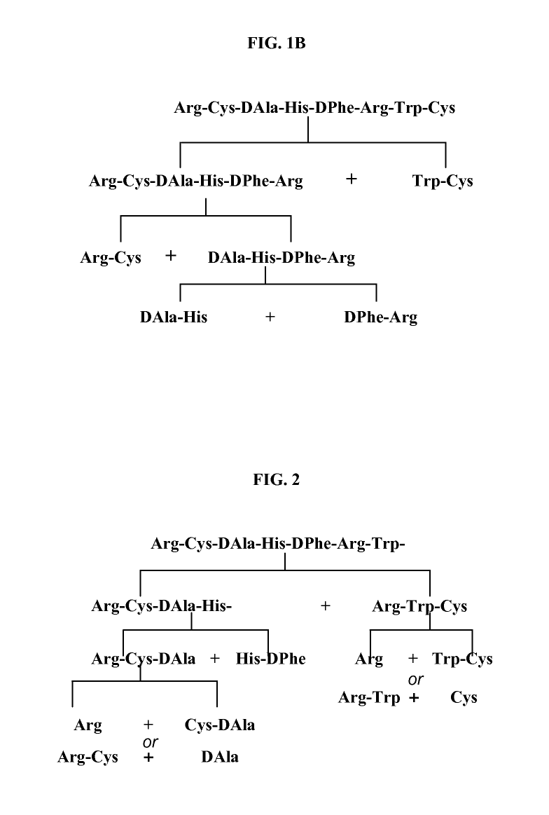

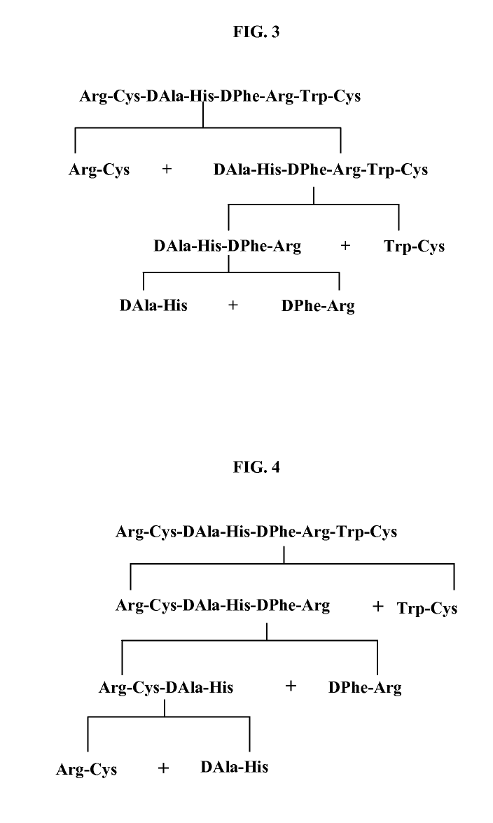

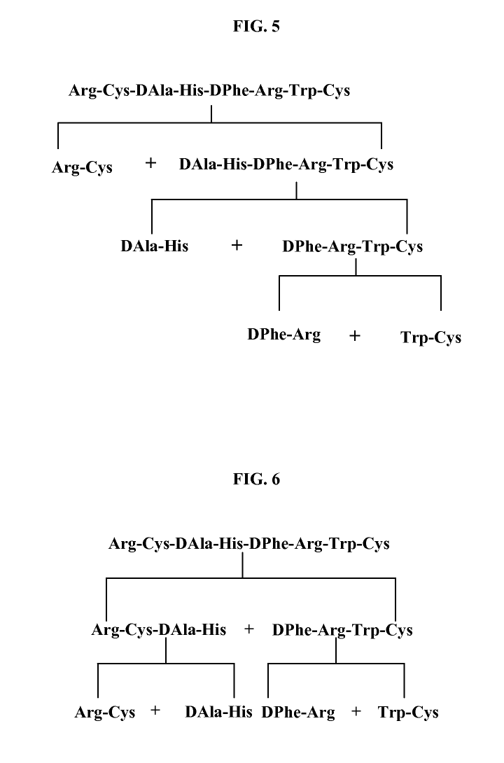

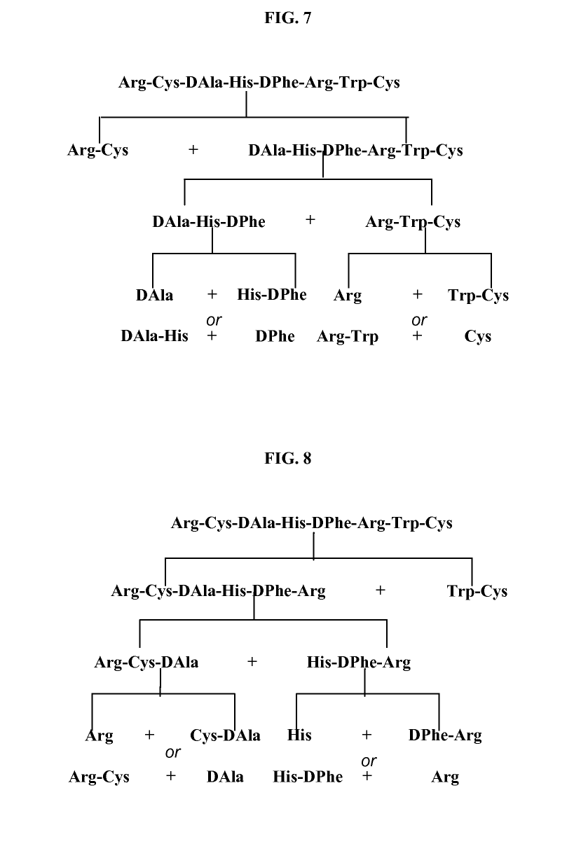

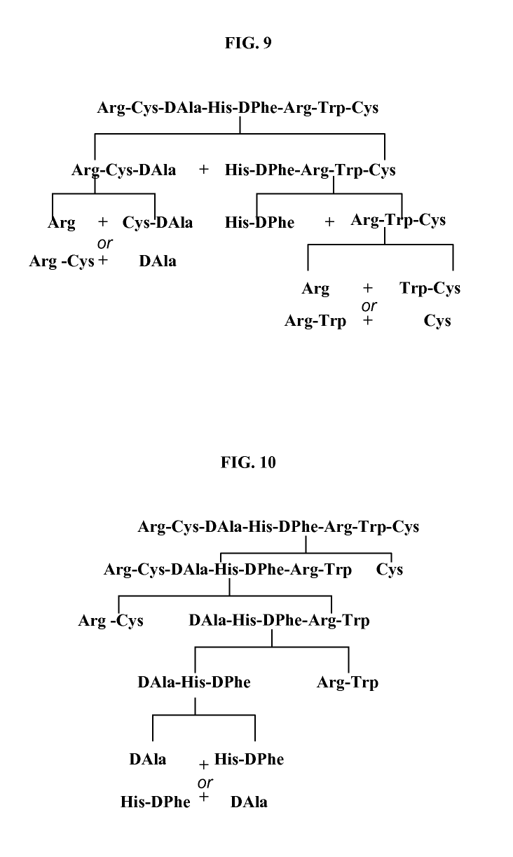

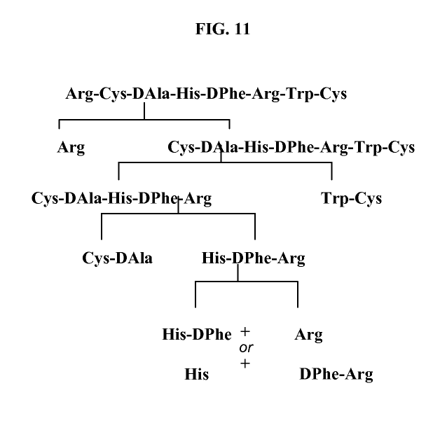

### DETAILED DESCRIPTION OF THE INVENTION

The application employs the following abbreviations:

Ac: acetyl
Acm: acetamidomethyl
AcOH: acetic acid
Ala or A: alanine
Arg or R: arginine
Boc: tert-butyloxycarbonyl
Bsmoc: 1,1-dioxobenzo[b]thiophene-2-ylmethyloxycarbonyl
BTFA: boron-tris-trifluoroacetate
Bzl: benzyl
Cys or C: cysteine
DCCI: N,N′-dicyclohexylcarbodiimide
DIC: N,N′-diisopropylcarbodiimide
DMF: dimethylformamide
EDC: 1-ethyl-3-(3-dimethylaminopropyl)carbodiimide hydrochloride

### Fmoc: 9-Fluorenylmethyloxycarbonyl

For: formyl
HATU: O-(7-azabenzotriazol-1-yl)-1,1,3,3-tetramethyluronium hexafluorophosphate
HBTU: 2-(1H-benzotriazol-1-yl)-1,1,3,3-tetramethyluronium hexafluorophosphate
His or H: histidine
HOBt 1-hydroxybenzotriazole
HPLC: high performance liquid chromatography
Me: methyl
MeOH: methanol
Mtr: 4-methoxy-2,3,6-trimethylbenzenesulfonyl
Mtt: methyltrityl
NH₂-CMe₂CP: dimethylcyclopropylmethyl amine
Pbf: 2,2,4,6,7-pentamethyldihydrobenzofuran-5-sulfonyl
Pmc: 2,2,5,7,8-pentamethylchroman-6-sulfonyl
TAEA: tris(2-aminoethyl)amine
TFA trifluoroacetic acid
THF: tetrahydrofuran
Trp or W: tryptophan
Trt: trityl
Z or Cbz: benzoyloxycarbonyl

The designation “NH₂” in Ac-Arg-cyclo(Cys-D-Ala-His-D-Phe-Arg-Trp-Cys)-NH₂ indicates that the C-terminus of the peptide is amidated.

The designation “-cyclo(Cys-Cys)-” indicates the structure:

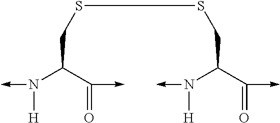

The term “about” as used herein, in associations with parameters and amounts, means that the parameter or amount is within ±5% of the stated parameter or amount.

### Synthesis

Synthesis of Example 1, i.e., Ac-Arg-cyclo(Cys-D-Ala-His-D-Phe-Arg-Trp-Cys)-NH₂
Step 1: Preparation of Z-D-Ala-His-OH

30 mmol of Z-D-Ala-OH in 150 mL of acetonitrile was cooled to −18° C. To the cooled solution, 3.4 mL of N-methyl-morpholine followed by 4.1 mL of chloroformic acid isobutyl ester were added. The solution was stirred for 15 minutes at −15° C. and a cold solution of 30.3 mmol of H-His-OH in 30 mL of IN NaOH and 100 mL of acetonitrile was added. The resulting solution was stirred for additional 18 hours at a temperature of −5° C. to 0° C. The reaction mixture was concentrated under vacuum, diluted with water, and extracted 3 times with a small quantity of ether. The aqueous phase was adjusted to about pH 2 by the addition of 4N H₂SO₄. A precipitate formed which was extracted with ether/acetic acid. The organic phase was washed with water and dried over Na₂SO₄.

After evaporation under vacuum, the residue was crystallized from ether/petrolether to yield the title compound.
Step 2: Preparation of Z-D-Phe-Arg(Pbf)-OMe

13.5 mL of chloroformic acid isobutyl ester was added, with stirring, to 147 mmol of Z-DPhe-OH dissolved in 1200 mL of THF which had been pre-cooled to −20° C. Stirring was continued for additional 20 minutes at −15° C. and a cold solution (−21° C.) of 158 mmol of HCl·H-Arg(Pbf)-OMe in 600 mL of THF was added. 17 mL of triethylamine was added dropwise and the temperature of the reaction vessel was maintained at a temperature of −15° C. The reaction mixture was stirred for 18 hours at 0° C. and the product was subjected to concentration under vacuum. Dilution was effected with ether/ethylacetate (1:1) and filtration was performed. The filtrate was washed with 2 N citric acid, 10% KHCO₃, and water. The organic phase was dried over Na₂SO₄ and evaporated to dryness to yield the title compound.
Step 3: Preparation of Z-D-Ala-His-D-Phe-Arg(Pbf)-OMe

131 mmol of Z-D-Phe-Arg(Pbf)-OMe in 150 mL of MeOH was hydrogenated in the presence of Pd/C. The product was filtered and washed with MeOH. The filtrate was evaporated under vacuum. The residue that formed was dissolved together with 99 mmol of Z-D-Ala-His-OH and 2.0 g HOBt in 50 mL of DMF. The solution was cooled to −15° C. 2.1 g of DCCI in 15 mL of DMF was added with stirring. The obtained solution was stirred for additional 2 days at 0° C., evaporated under vacuum, diluted with acetic acid/ether (1:1), and filtered. The filtrate was washed with 2 N citric acid, 10% KHCO₃ and water. The organic phase was dried over Na₂SO₄ and evaporated. The residue was purified by chromatography over silica gel with CH₂Cl₂/MeOH as an eluant. The fractions containing the desired product were collected and evaporated under vacuum to yield the title compound.
Step 4: Preparation of H-D-Ala-His-D-Phe-Arg(Pbf)-OMe 1.3 mmol of Z-D-Ala-His-D-Phe-Arg(Pbf)-OMe in 50 mL of MeOH was hydrogenated in the presence of 10% Pd/C and the solution was filtered and evaporated under vacuum to yield the title compound.
Step 5: Preparation of Ac-Arg(Pbf)-Cvs(Acm)-OH

8.4 mL of NEt₃, followed by 8.3 mL of chloroformic acid isobutyl ester, were added to 60 mmol of Ac-Arg(Pbf)-OH in 300 mL of THF that was pre-cooled to −20° C. The obtained mixture was stirred for 10 minutes at −15° C. A cold solution of H-Cys(Acm)-OH and 11.5 mL of NEt₃ in 400 mL of THF/water (5:1) was subsequently added dropwise. The reaction mixture was stirred for about 2 days at 0° C., concentrated under vacuum, diluted with 1.3 L of water, and extracted with ether. The aqueous phase was adjusted to pH 2.0 by the addition of 4 N H₂SO₄, and the precipitate was extracted with ethyl acetate. The ethyl acetate extract was washed with water, dried over Na₂SO₄ and evaporated under vacuum. The residue was purified by chromatography over silica gel with ether/1% AcOH as an eluant. The fractions containing the desired product were combined and evaporated under vacuum to yield the title compound.
Step 6: Preparation of Ac-Arg(Pbf)-Cys(Acm)-D-Ala-His-D-Phe-Arg(Pbf)-OMe

0.30 g of DCCI was added with stirring to 1.42 mmol of Ac-Arg(Pbf)-Cys(Acm)-OH, 1.3 mmol of H-D-Ala-His-D-Phe-Arg(Pbf)-OMe and 0.5 g of HOBt dissolved in 30 mL of DMF pre-cooled to −20° C. The reaction mixture was stirred for about 12 hours at a constant temperature of −5° C. to 0° C. and then for about 4 hours at room temperature. The precipitated dicyclohexyl urea was filtered off and the filtrate was washed with 2 N citric acid, 10% KHCO₃ and water. The organic phase was dried over Na₂SO₄, purified and concentrated. The product was precipitated by the addition of ether, filtered and dried to yield the title compound.
Step 7: Preparation of Ac-Arg(Pbf)-Cys(Acm)-D-Ala-His-D-Phe-Arg(Pbf)-NHNH₂

1.5 mL of hydrazine hydrate was added to 0.07 mmol of Ac-Arg(Pbf)-Cys(Acm)-D-Ala-His-D-Phe-Arg(Pbf)-OMe dissolved in 15 mL of DMF. The solution was allowed to stand for 5 hours at room temperature. Aqueous MeOH was then added. The precipitate that formed was filtered off, washed with MeOH/water (1:9), and dried to yield the title compound.
Step 8: Preparation of H-Trp-Cys(Acm)-OMe

A solution of 17.2 mmol of Boc-Trp-Cys(Acm)-OMe and 5 mL of thioanisole in 25 mL of methylenechloride was added to 50 mL of TFA and allowed to stand for 20 minutes at room temperature. The solution was diluted with about 1.5 L of ether, and the precipitate that formed was filtered off, washed with ether and dried to yield the title compound.
Step 9: Preparation of Boc-Trp-Cys(Acm)-OMe

2.1 mL of N-methylmorpholine was added with stirring to 19.4 mmol of Boc-Trp-OH in 50 mL of THF pre-cooled to −20° C. followed by dropwise addition at −15° C. of 2.4 mL of chloroformic acid isobutyl ester. After stirring for 5 minutes at −15° C., a cold solution (−10° C.) comprising 24 mmol of H-Cys(Acm)-OMe and 4.1 mL of N-methylmorpholine in 30 mL of DMF was added. The mixture was stirred for 2 hours at a constant temperature of 0° C. and then for an additional 2 hours at room temperature. 50 mL of 10% KHCO₃ was added. The reaction mixture was concentrated under vacuum, diluted with ethyl acetate and washed 3 times with 2 N citric acid, then 3 times with 10% KHCO₃, and then with a 30% NaCl solution. The organic phase was dried over Na₂SO₄ and evaporated under vacuum to yield the title compound.
Step 10: Preparation of Ac-Arg(Pbf)-Cys(Acm)-D-Ala-His-D-Phe-Arg(Pbf)-Trp-Cys(Acm)-OMe

0.7 mL of 5 N HCl in ether was added with stilling to 0.63 mmol of Ac-Arg(Pbf)-Cys(Acm)-D-Ala-His-D-Phe-Arg(Pbf)-NHNH₂ in 30 mL of DMF pre-cooled to −20° C. 0.8 mL of 10% tert-butylnitrite in DMF was then added. The mixture was stirred for 15 minutes at −20° C. to −15° C. 0.56 mL of NEt₃ was added at −25° C. followed by a solution of 1.1 mmol of H-Trp-Cys(Acm)-OMe in 3 mL of DMF pre-cooled to −15° C. and the resulting mixture was stirred for 20 hours at −5° C. to 0° C. and then for additional 3 hours at room temperature. The reaction mixture was diluted with about 100 mL of methanol and the product was precipitated by the addition of approximately 40 mL of water. The precipitated that formed was filtered off, washed with aqueous MeOH and dried to yield the title compound.
Step 11: Preparation of Ac-Arg(Pbf)-Cys(Acm)-D-Ala-His-D-Phe-Arg(Pbf)-Trp-Cys(Acm)-NH₂

0.5 mmol of Ac-Arg(Pbf)-Cys(Acm)-D-Ala-His-D-Phe-Arg(Pbf)-Trp-Cys(Acm)-OMe was treated with liquid ammonia for several hours. The ammonia was then slowly evaporated. The residue was diluted with about 100 mL of methanol and the product was precipitated out by the addition of approximately 40 mL of water. The precipitate that formed was filtered off, washed with aqueous MeOH and dried to yield the title compound.
Step 12: Preparation of Ac-Arg-cyclo(Cys-D-Ala-His-D-Phe-Arg-Trp-Cys)-NH₂

0.4 mmol of Ac-Arg(Pbf)-Cys(Acm)-D-Ala-His-D-Phe-Arg(Pbf)-Trp-Cys(Acm)-NH₂ and 6.0 mL of thioanisol were dissolved in 30 mL of TFA at 0° C. Stirring was continued for 1.5 hours maintaining a temperature of −5° C. to −10° C. and subsequently 50 mL of cold MeOH (−70° C.) was added. After additional 5 minutes, 1 L of ether and 5 mL of approximately 5 N HCl in ether were added with stirring. The precipitated product was filtered off and washed briefly with ether. The residue was dissolved immediately in 2.5 L of MeOH/H₂O. The pH was adjusted to 7-7.5 by the addition of a dilute ammonia solution. To the mixture was added iodine solution in MeOH dropwise until a persistent color was achieved. The reaction was stirred at room temperature until it tested negative for -SH groups (e.g., the Ellmann test). The reaction was quenched with 10% NaS₂O₃. The pH was adjusted to 3-4 by the addition of HCl. The obtained solution was concentrated under vacuum and the product was lyophilized. The lyophilisate was dissolved in a small quantity of 10% AcOH and purified by HPLC. Fractions containing the desired product were collected and lyophilized to yield the title compound.

Additional embodiments of the present invention will be apparent from the foregoing disclosure and are intended to be encompassed by the invention as described fully herein and defined in the following claims.

What is claimed is:

1. A process for the solution-phase synthesis of Ac-Arg-cyclo(Cys-D-Ala-His-D-Phe-Arg-Trp-Cys)-NH2 comprising a fragment condensation procedure.

2. A process for the solution-phase synthesis of Ac-Arg-cyclo(Cys-D-Ala-His-D-Phe-Arg-Trp-Cys)-NH2 according to claim 1, wherein protected amino acids are used.

3. A process for the solution-phase synthesis of Ac-Arg-cyclo(Cys-D-Ala-His-D-Phe-Arg-Trp-Cys)-NH2 according to claim 2, wherein said protected amino acids are selected from the group consisting of Boc protected amino acids, benzyloxycarbonyl protected amino acids, Fmoc protected amino acids, and protected amino acid fluorides.

4. A process for the solution-phase synthesis of Ac-Arg-cyclo(Cys-D-Ala-His-D-Phe-Arg-Trp-Cys)-NH2 according to claim 3, wherein said protected amino acid fluoride is Fmoc amino acid fluoride or Bsmoc amino acid fluoride.

5. A process for the solution-phase synthesis of Ac-Arg-cyclo(Cys-D-Ala-His-D-Phe-Arg-Trp-Cys)-NH2 according to claim 1, wherein a mixed anhydride coupling method is employed.

6. A process for the solution-phase synthesis of Ac-Arg-cyclo(Cys-D-Ala-His-D-Phe-Arg-Trp-Cys)-NH2 according to claim 1, wherein a peptide-hydrazide coupling method is employed.

7. A process for the solution-phase synthesis of Ac-Arg-cyclo(Cys-D-Ala-His-D-Phe-Arg-Trp-Cys)-NH2 according to claim 1, wherein ammonia is used to convert an ester functional group to an amide functional group.

8. A process for the solution-phase synthesis of Ac-Arg-cyclo(Cys-D-Ala-His-D-Phe-Arg-Trp-Cys)-NH2 according to claim 1, wherein a protected peptide fragment Trp-Cys or Arg-Trp is provided.

9. A process for the solution-phase synthesis of Ac-Arg-cyclo(Cys-D-Ala-His-D-Phe-Arg-Trp-Cys)-NH2 according to claim 8, wherein said protected peptide fragment Trp-Cys is Boc-Trp-Cys(Acm)-OMe or Boc-Trp(For)-Cys(Acm)-OMe.

10. A process for the solution-phase synthesis of Ac-Arg-cyclo(Cys-D-Ala-His-D-Phe-Arg-Trp-Cys)-NH2 according to claim 8, wherein a protected peptide fragment D-Ala-His or His-D-Phe is provided.

11. A process for the solution-phase synthesis of Ac-Arg-cyclo(Cys-D-Ala-His-D-Phe-Arg-Trp-Cys)-NH2 according to claim 10, wherein said protected peptide fragment D-Ala-His is bcnzoyloxycarbonyl benzyloxycarbonyl-D-Ala-His-OH or benzyloxycarbonyl-D-Ala-His(Trt)-OH.

12. A process for the solution-phase synthesis of Ac-Arg-cyclo(Cys-D-Ala-His-D-Phe-Arg-Trp-Cys)-NH2 according to claim 1, comprising the steps of:
(a) synthesizing a fragment benzyloxycarbonyl-D-Ala-His(Trt)-OH from benzyloxycarbonyl-D-Ala-OH and H-His(Trt)-OH in the presence of a coupling reagent;
(b-1) synthesizing a fragment benzyloxycarbonyl-D-Phe-Arg(Pbf)-OMe from benzyloxycarbonyl-D-Phe-OH and H-Arg(Pbf)-OMe in the presence of a coupling reagent;
(b-2) synthesizing a fragment H-D-Phe-Arg(Pbf)-OMe by hydrogenating the fragment benzyloxycarbonyl-D-Phe-Arg(Pbf)-OMe obtained in the step (b-1);
(c-1) synthesizing a fragment benzyloxycarbonyl-D-Ala-His(Trt)-D-Phe-Arg(Pbf)-OMe from benzyloxycarbonyl-D-Ala-His(Trt)-OH and the fragment H-D-Phe-Arg(Pbf)-OMe obtained in the step (b-2) in the presence of a coupling reagent;
(c-2)
synthesizing a fragment H-D-Ala-His(Trt)-D-Phe-Arg(Pbf)-OMe by hydrogenating the fragment bcnzoyloxycarbonyl benzyloxycarbonyl-D-Ala-His(Trt)-D-Phe-Arg(Pbf)-OMe obtained in the step (c-1);
(d-1) or alternatively, synthesizing a fragment Ac-Arg(Pbf)-Cys(Acm)-OH from Ac-Arg(Pbf)-OH and H-Cys(Acm)-OMe in the presence of a coupling reagent, followed by hydrolysis by using a base;
(e-1) synthesizing a fragment Ac-Arg(Pbf)-Cys(Acm)-D-Ala-His(Trt)-D-Phe-Arg(Pbf)-OMe from Ac-Arg(Pbf)-Cys(Acm)-OH and H-D-Ala-His(Trt)-D-Phe-Arg(Pbf)-OMe in the presence of a coupling reagent;
(f) synthesizing a fragment Ac-Arg(Pbf)-Cys(Acm)-D-Ala-His(Trt)-D-Phe-Arg(Pbf)-NHNH2 from the fragment Ac-Arg(Pbf)-Cys(Acm)-D-Ala-His(Trt)-D-Phe-Arg(Pbf)-OMe obtained in the step (e-1) in the presence of hydrazine;
(g-1) synthesizing a fragment Boc-Trp-Cys(Acm)-OMe from Boc-Trp-OH and H-Cys(Acm)-OMe in the presence of a coupling reagent;
(g-2) synthesizing H-Trp-Cys(Acm)-OMe from the fragment Boc-Trp-Cys(Acm)-OMe obtained in the step (g-1) in the presence of TFA;
synthesizing Ac-Arg(Pbf)-Cys(Acm)-D-Ala-His(Trt)-D-Phe-Arg(Pbf)-Trp-Cys(Acm)-OMe from the fragment Ac-Arg(Pbf)-Cys(Acm)-D-Ala-His(Trt)-D-Phe-Arg(Pbf)-NHNH2 obtained in the step (f) and H-Trp-Cys(Acm)-OMe by using an acid and tert-butylnitrite;
synthesizing Ac-Arg(Pbf)-Cys(Acm)-D-Ala-His(Trt)-D-Phe-Arg(Pbf)-Trp-Cys(Acm)-NH2 from Ac-Arg(Pbf)-Cys(Acm)-D-Ala-His(Trt)-D-Phe-Arg(Pbf)-Trp-Cys(Acm)-OMe obtained in the step (h) in the presence of ammonia;
(i-1) deprotecting the His residue in Ac-Arg(Pbf)-Cys(Acm)-D-Ala-His(Trt)-D-Phe-Arg(Pbf)-Trp-Cys(Acm)-NH2 in the presence of TFA; and
(j) synthesizing Ac-Arg-cyclo(Cys-D-Ala-His-D-Phe-Arg-Trp-Cys)-NH2 from Ac-Arg-Cys(Acm)-D-Ala-His-D-Phe-Arg-Trp-Cys(Acm)-NH2 by using an oxidizing agent.

13. A process for the solution-phase synthesis of Ac-Arg-cyclo(Cys-D-Ala-His-D-Phe-Arg-Trp-Cys)-NH2 according claim 12, wherein:
said oxidizing agent is iodine;
said coupling reagent is DCC, HBTU, HATU, DIC, EDC, or chloroformic acid isobutyl ester; and
said base is Et2NH, TAEA, piperazine, sodium hydroxide, or potassium hydroxide.

14. A process for the solution-phase synthesis of Ac-Arg-cyclo(Cys-D-Ala-His-D-Phe-Arg-Trp-Cys)-NH2 according to claim 8, wherein
a protected peptide fragment Cys-D-Ala or Arg-Cys is provided.

15. A process for the solution-phase synthesis of Ac-Arg-cyclo(Cys-D-Ala-His-D-Phe-Arg-Trp-Cys)-NH2 according to claim 14, wherein said protected peptide fragment Cys-D-Ala is Boc-Cys(Acm)-D-Ala-OH.

16-25. (canceled)

26. A process for the solution-phase synthesis of Ac-Arg-cyclo(Cys-D-Ala-His-D-Phe-Arg-Trp-Cys)-NH2 according to claim 9, wherein a protected peptide fragment D-Ala-His or His-D-Phe is provided.

27. A process for the solution-phase synthesis of Ac-Arg-cyclo(Cys-D-Ala-His-D-Phe-Arg-Trp-Cys)-NH2 according to claim 26, wherein said protected peptide fragment D-Ala-His is benzoyloxycarbonyl-D-Ala-His-OH or benzoyloxycarbonyl-D-Ala-His(Trt)-OH.

28. A process for the solution-phase synthesis of Ac-Arg-cyclo(Cys-D-Ala-His-D-Phe-Arg-Trp-Cys)-NH2 according to claim 9, wherein a protected peptide fragment Cys-D-Ala or Arg-Cys is provided.
